(19) United States
(12) Patent Application Publication
(10) Pub. No.: US 2026/0249137 A1
(43) Pub. Date: Aug. 27, 2026

(54) REAL-TIME MODIFICATION OF WORKOUTS WITHIN A CONNECTED FITNESS PLATFORM

(71) Applicant: Peloton Interactive, Inc., New York, NY (US)

(72) Inventor: Francis SHANAHAN, Watchung, NJ (US)

(21) Appl. No.: 19/065,891

(22) Filed: Feb. 27, 2025

(51) Int. Cl. A63B 24/00; G16H 20/30

(52) U.S. Cl. CPC A63B 24/0075; A63B 24/0062; G16H 20/30

(57) ABSTRACT

The systems and methods described herein present a virtual coach and/or real-time modified workout that tailors, in real-time (or near real-time), an ongoing workout as the user progresses through different segments of the workout. For example, a workout system may guide, prompt, or otherwise cause a user to experience different branches or paths of an overall workout based on their needs, interest, or performance (e.g., metrics captured by sensors) during the workout and/or during an overall training program or plan. Further, the workout system may modify voice instructions presented to the user during a workout, such as when the user progresses between workout segments.

### CROSS-REFERENCE TO RELATED APPLICATIONS

This application is related to U.S. patent application Ser. No. _____, attorney docket No. 738MS0070US, filed on Feb. 27, 2025, entitled REAL-TIME MODIFICATION OF AUDIO CONTENT FOR A VIRTUAL COACH APPLICATION, which is hereby incorporated by reference in its entirety.

### BACKGROUND

The world of connected fitness is an ever-expanding one. This world can include a user taking part in an exercise activity or workout (e.g., running, cycling, lifting weights, rowing, and so on), as well as other users performing similar exercise activities or workouts. For some modalities, the users perform the exercise activities using a fitness machine (e.g., a treadmill, a stationary bike, a strength machine, a stationary rower, and so on). In other modalities, the users perform the exercise activities by moving through the world (e.g., by running, bicycling, rowing, and so on) and/or by exercising without a machine, such as strength training, doing yoga, stretching, hiking, climbing, and so on.

The users can have wearable devices or mobile devices (e.g., heart rate monitors, GPS trackers, fitness watches, and so on) that monitor their activity or performance. In some cases, such as in tandem with an exercise machine, the users may perform an exercise activity in front of a user interface (e.g., a display or device) that presents content (e.g., audio and/or video) associated with the exercise activity.

For example, the user interface, whether a mobile device, a display device, or a display that is part of a machine, can provide or present interactive content to the users. For example, the user interface can present live or recorded classes (e.g., fitness or strength classes), audio instructions, video tutorials of activities, workout plans (e.g., a list of movements), leaderboards and other competitive or interactive features, progress indicators (e.g., via time, distance, and other metrics), and so on.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIG. 2 is a block diagram illustrating components of a workout system.

FIG. 3 is a diagram illustrating a workout path modified for a user performing an exercise activity.

FIG. 4A is a flow diagram illustrating an example method for modifying a workout for a user of a connected fitness platform.

FIG. 4B is a flow diagram illustrating another example method for modifying a workout for a user of a connected fitness platform.

FIG. 5 is a flow diagram illustrating an example method for presenting voice instructions to a user of a connected fitness platform.

FIG. 6 is a flow diagram illustrating an example method for coaching a user of a connected fitness platform.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods may perform real-time and/or personalized modifications for users performing exercise activities, such as exercise activities associated with a connected fitness platform. For example, the connected fitness platform may stream an instructor-led exercise class to a user (e.g., riding an exercise bicycle and/or running on a treadmill) and/or may act as or provide a virtual coach as the user progresses through a workout (e.g., an interval run or strength workout), providing voice instructions to the user during the workout (e.g., via a mobile application on the user's mobile device).

Typical user experiences within a connected fitness environment involve a linear progression through a workout (e.g., an instructor-led workout). Workout parameters (e.g., class segments, duration, sequence, and so on) are pre-determined before a user begins a class, a training sequence, or other exercise activity. Thus, while the user may receive a personalized workout (e.g., classes selected for the user based on a variety of factors), the workout is often static and inflexible once the user commences performance.

The systems and methods describe herein introduce technology that mimics or represents virtual coaching within a workout presented to a user, such as a user of a connected fitness platform. For example, a connected fitness platform may insert and/or define infection points or branches into a layout or structure of a workout (e.g., at various stages of a sequence of workout segments that combine to form a workout or exercise activity). The inflection points or branches may tailor, in real-time (or near real-time), an ongoing workout as the user progresses through the different segments of the workout.

Thus, the layout or structure of the workout may include several branches of workout segments. The user may be guided or prompted to experience different branches of the overall workout based on their (current) needs, interest, or performance (e.g., metrics captured by sensors) during the workout and/or during an overall training program or plan.

In some embodiments, the technology may modify voice instructions presented to the user during a workout, such as when the user progresses between workout segments. For example, as the user completes a workout segment of an instructor-led workout (e.g., the instructor acting a virtual coach), the connected fitness platform modified voice instructions to be presented to the user based on a real-time assessment of the user's performance or exercise activity. For example, the platform may access an large language model (LLM) that generates voice transcripts for the instructor and modify, generate, or update the voice transcript underlying a voice instruction next to be presented to the user.

Thus, the systems and methods described herein can provide users of a connected fitness platform with real-time coaching during a workout or exercise activity. The systems and methods can provide users with a “choose your own adventure” style workout program based on their performance, interest, or other factors, as well as a virtual coach that adapts its instructions (e.g., voice cues, prompts, encouragement, and so on) to an ever-changing performance by a user, mimicking a real-life coach, among other benefits.

Various embodiments of the system and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

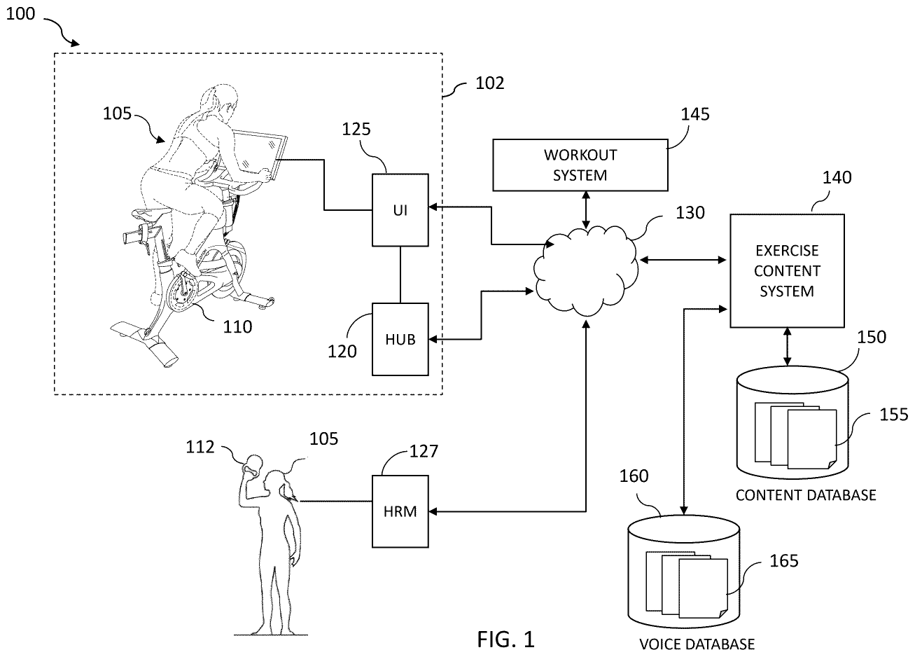

The technology described herein is directed, in some embodiments, to providing a user with an enhanced user experience (e.g., a modified, in real-time, workout experience) when performing an exercise activity or workout, such as a sequence, group, set, or block of exercise activities as part of a connected fitness system or other exercise system. FIG. 1 is a block diagram illustrating a suitable network environment 100 for users of an exercise system.

The network environment 100 includes an activity environment 102, where a user 105 is performing an exercise activity, such as an indoor cycling activity. In some cases, the user 105 can perform the activity with an exercise machine 110, such as an exercise bicycle, a treadmill, a rowing machine, a stair climber, and so on.

In some cases, the exercise activity performed by the user 105 can include a variety of different workouts, activities, actions, and/or movements not associated with a machine, such as movements associated with lifting weights 112 (as shown), stretching, meditation, yoga, Pilates, rowing, running, cycling, jumping, sports movements (e.g., throwing a ball, pitching a ball, hitting, swinging a racket, swinging a golf club, kicking a ball, hitting a puck), and so on.

The activity environment 102 may include a home or home gym of the user, a commercial fitness center or gym, a hotel, a specific sports facility (e.g., yoga studio, batting cage, indoor track, and so on), an outdoor venue (e.g., a track, running path, street, park, and so on) or other workout facility or location that includes exercise machines, workout devices or accessories (e.g., weights, heavy bags, and so on). The exercise system, or platform, may access, obtain, and/or store information about the facility/location, such as the types of exercise machines or activities available to users, the hours available to utilize the facility/location, and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., streamed classes, dynamically changing video, audio, video games, instructional content, workout instructions, and so on) to the user 105 during an activity or workout.

The exercise machine 110, in some embodiments, can include a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box, mounted screen, or other similar device that outputs signals to a display, such as the user interface 125. Thus, the media hub 120 can operate to both capture images of the user 105 during an activity, while also presenting content (e.g., time-or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

Further, in various embodiments, the media hub 120 can facilitate the tracking of movements or actions performed by the user 105, such as when the user is performing different movements as part of a strength or lifting class. The media hub 120, as described herein, can track and/or identify movements performed by the user 120.

In some cases, a heart rate monitor (HRM) 127 or other wearable device (e.g., smart watches, headphones, fitness trackers, augmented reality, virtual reality, extended reality (AR/VR/XR) displays or devices, GPS devices, smart glasses, and so on) can capture information about the user 105, such as heart rate and other biometric information, movement information (e.g., speed, distance, or acceleration information), sleep information, and so on. The HRM 127 can capture the user's heart rate and other information during machine-based activities and/or other activities, such as offline or class-based activities that do not utilize the exercise machine 110. In some cases, the exercise machine 110 can include components configured to capture biometric information for the user 105, such as heart rate information. Further, the HRM 127 or smart watch may also provide a user interface (e.g., the user interface 125) to the user.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) an exercise content system 140 over the network 130. In other cases, the media hub 120 controls the communication of content to/from the exercise content system 140 over the network 130 and presents the content to the user via the user interface 125.

The exercise content system 140, located at one or more servers remote from the user 105, can include various content libraries (e.g., classes, movements, tutorials, and so on) and perform functions to stream or otherwise send content to the machine 110, the media hub 120, and/or the user interface 125 over the network 130.

A content database 150 stores content 155 (e.g., video files) that presents a pre-recorded class to a user. The content can include images, video, and other visual information that present the class, music and other audio information to be played during the activity, and various overlay or augmentation information that is presented along with the audio/video content. Further, the database 150 can include various content libraries (e.g., classes, movements, tutorials, and so on) associated with the content presented to the user during a selected experience.

A voice database 160 stores voice or audio content 165, such as voice instructions generated by instructors or coaches within the connected fitness platform. In some cases, the voice or audio content 165 may be based on, or generated, by one or more large language models (LLMs), such as LLMs associated with each of the instructors. For example, the content 165 may include voice transcripts that support and/or are associated with voice instructions (e.g., commands, prompts, cues, encouragement, and so on) spoken by instructions of the platform. LLMs, such as generative AI models (e.g., GPT-4, PaLM2, and so on), may generate class or voice transcripts for different instructors. The LLMs may be trained by other content associated with the instructors, such as the content 155 (e.g., recorded exercise classes or audio training programs led by the instructors).

In some cases, the content may include simple or targeted workout plans or activities that include lists of movements or activities (e.g., perform 3 sets of a certain movement or run for 10 minutes at medium pace), and is not associated with video or audio classes or content. For example, a workout plan may be based on three or four different workout segments, where each segment is associated with an instructor and a voice cue or instruction provided by the instructor.

Further, in some cases, aspects of the content may be dynamically generated during an activity, such as during a running class or activity. The content may be generated in response to the user performing the activity, in response to the user moving through an environment (e.g., a real or virtual environment), and so on.

As described herein, a workout system 145 can include various components configured to present a virtual coach and/or real-time modified workout that tailors, in real-time (or near real-time), an ongoing workout as the user progresses through different segments of the workout. For example, the workout system 145 may guide, prompt, or otherwise cause the user 105 to experience different branches or paths of an overall workout based on their (current) needs, interest, or performance (e.g., metrics captured by sensors) during the workout and/or during an overall training program or plan. Further, the workout system 145 may modify or generate voice instructions presented to the user during a workout, such as when the user progresses between workout segments. Further details regarding the workout system 145 are described herein.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network 130, or cloud, can be any network, such as a wired or wireless local area network (LAN), a wired or wireless wide area network (WAN), the Internet, some other public or private network, a cellular (e.g., 4G, LTE, 5G, 6G network), and so on. While the connections between the various devices and the network 130 and are shown as separate connections, these connections can be any kind of local, wide area, wired, or wireless network, public or private.

Further, any or all components depicted in the Figures described herein can be supported and/or implemented via one or more computing systems, services (e.g., cloud instances), or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of Modifying Workouts for Users

The connected fitness platform, as described herein, may employ a workout system, such as the workout system 145, to modify and/or enhance an ongoing workout or exercise activity for a user. As described herein, the workout may be a recorded (or partially recorded) exercise class that is streamed to the user via the user's exercise bicycle or treadmill and/or may be an instructor-led or -guided workout that is independent of any class or structure activity, such as a sequence of movements, segments, intervals, sets, or other activities.

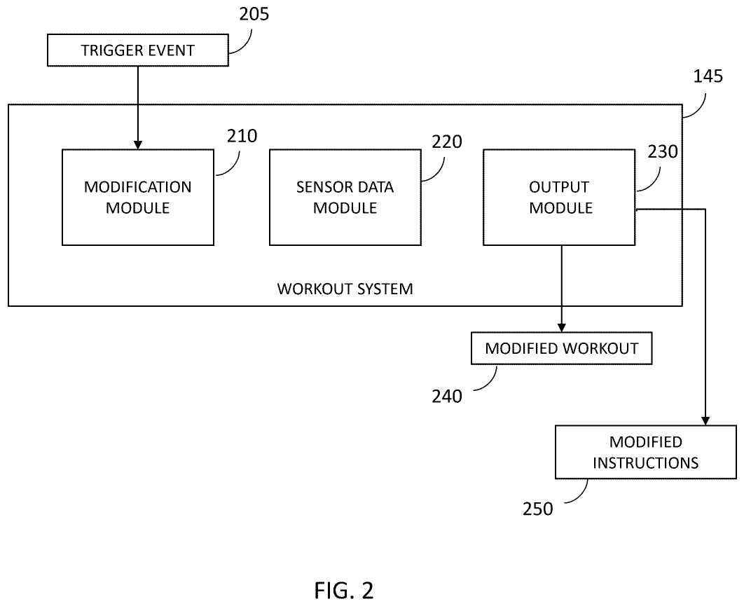

FIG. 2 is a block diagram illustrating example modules of the workout system 145. The workout system 145, which can be a system of components, modules, or devices, functions to modify or tailor content (e.g., video, audio, and so on) presented to users, such as members of the connected fitness platform, a fitness service, a virtual coaching program, and so on.

The components and/or modules of the workout system 145 can be implemented with a combination of software (e.g., executable instructions, or computer code) and hardware (e.g., at least a memory and processor). Accordingly, as used herein, in some embodiments, a component/module is a processor-implemented component/module and represents a computing device having a processor that is at least temporarily configured and/or programmed by executable instructions stored in memory to perform one or more of the functions that are described herein. The workout system 145 includes a modification module 210, a sensor data module 220, and an output module 230.

In some embodiments, the modification module 210 is configured and/or programmed to receive a trigger event 205 within a workout performed by a user (e.g., tracked by the connected fitness platform). For example, the trigger event 205 may be any event or occurrence during a workout, during a workout segment, and/or during a time period between workout segments.

The trigger event 205 may include the user completing a segment (or nearing completion of the segment), the user starting a segment, the user selecting a presented workout option, performance metrics (e.g., heart rate, exercise-specific metrics, and so on) being within and/or outside of a threshold or range of expected metrics, and so on.

In some embodiments, the sensor data module 220 is configured and/or programmed to access sensor data representing one or more workout metrics associated with the workout performed by the user in response to the trigger event. The sensor data may include heart rate data and/or other biometrics for the user while performing a current workout segment of the workout (e.g., captured by a wearable device), performance metrics (e.g., speed, distance, acceleration) associated with the user performing a current workout segment of the workout (e.g., captured by the wearable device and/or an associated exercise machine), and so on.

For example, a user may be performing a cycling activity via an exercise bicycle that follows an instructor-led exercise class streamed to the user via display of the exercise bicycle. The class has multiple segments (e.g., a warmup, cool down, and multiple effort sections). During the class, the exercise bicycle, a smart watch (or another wearable device) may capture heart rate information and/or performance metrics, such as cadence, resistance, output, calories burned, effort, and so on.

As another example, the user is running outside performing a series of intervals (e.g., short duration sprints), being coached by voiced instructions (e.g., and/or closed-captioned instructions) from an instructor of the workout. During the class, the user's smart watch captures their heart rate and/or performance metrics, such as speed, distance, time, elevation gain, calories burned, and so on.

In some embodiments, the output module 230 is configured and/or programmed to generate and/or present a modified workout 240 based on the one or more workout metrics for the workout performed by the user. The output module 230 may modify a path of workout segments presented to the user within the workout based on the one or more workout metrics not satisfying threshold metrics for the path of workout segments, may insert a workout segment into a path of workout segments presented to the user within the workout based on the one or more workout metrics not satisfying threshold metrics for the path of workout segments, may modify a next workout segment of a path of workout segments presented to the user within the workout based on the one or more workout metrics, and so on.

For example, the output module 230 may replace a next workout segment with another workout segment based on a real-time assessment, via the workout metrics, of the user performing the workout. In some cases, the output module 230 may insert a workout segment, remove a workout segment, modify a workout segment (e.g., shorten or lengthen the segment), or otherwise modify the workout based on the real-time assessment. Further, in some cases, the output module 230 may generate modified instructions (e.g., voice or text instructions) 250 that are presented (e.g., played) to the user during the workout, as described herein.

As described herein, in some cases, the workout may include multiple branching paths of workout segments (e.g., sub-classes, movements, and so on), and the user may select various paths through the workout. In some cases, the trigger event 205 is a selection by the user of one or more candidate workout segments of the workout (e.g., where the candidate segments are selected or generated by the performance metrics).

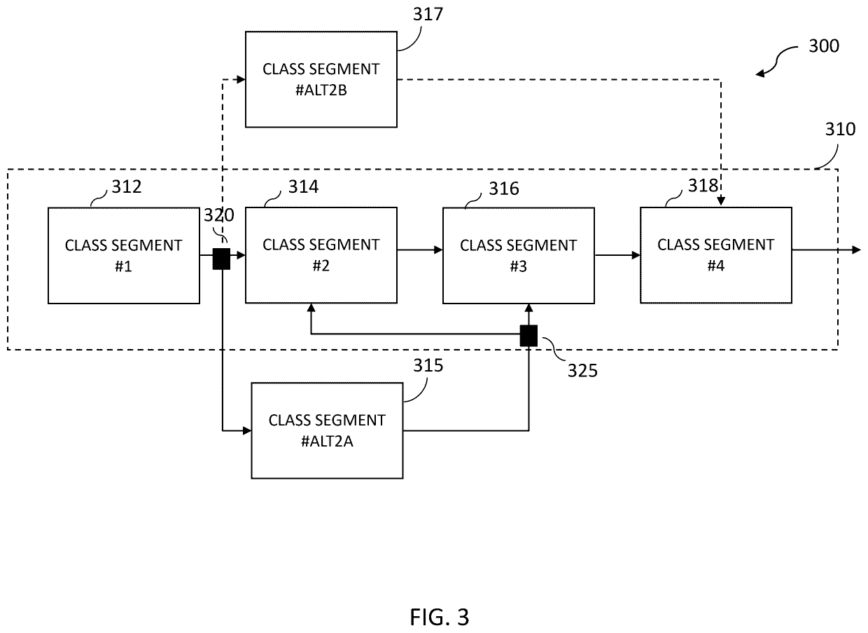

FIG. 3 is a diagram illustrating a workout path 300 that is modified for a user performing an exercise activity. The user may begin the workout by performing an initial workout segment 312 (e.g., class segment #1) of a baseline path 310 that includes the initial workout segment 312 and next workout segments 314, 316, 318 (e.g., class segments #2-#4). In some cases, the baseline path 310 is personalized for the user before commencement of the workout.

After completing the initial workout segment 312, the user traverses to an inflection point 320 (or branch point), where the workout system 145 may present user-selectable workout options for the user and/or may automatically select a next workout segment (or branch through the workout).

As a first example, the workout system 145 may present (e.g., in audio or video form), a series of user-selectable workout options for the user to take the next workout segment 314 (class segment #2) or multiple alternative class segments 315, 317 (e.g., class segments #ALT2A and #ALT2B). The following exercise scenario illustrates the workout system 145 presenting workout options to the user during a workout:

The user is running on a treadmill and has just finished a warmup section (e.g., class segment #1) of a streaming exercise class. The class progresses to the inflection point 320 (e.g., a trigger event 205), causing the class to present, via a display of the treadmill) the following options:

“Are you ready to go?” (e.g., an option for the next class segment #2 of the baseline path 310);

“Do you need more warmup time?” (e.g., class segment #ALT2A, allowing the user to continue to warmup);

“Do you want to stretch a bit?” (e.g., class segment #ALT2B, moving the user to an active stretching activity before entering later running segments); and so on.

As another example, the user progresses to an inflection point 325, after moving through the initial workout segment 312 and the alternative workout segment 315. The workout system 145 may automatically select the next workout segment for the user (e.g., class segment #2 or class segment #3), based on various metrics, as described herein. The following exercise scenario illustrates the workout system 145 dynamically and/or automatically selecting workout segments for the user:

The running on a treadmill and has just finished the warmup section (e.g., class segment #1) of the streaming exercise class and the alternative workout segment 315. The class progresses to the inflection point 325 (e.g., a trigger event 205), and determines, based on the user's heart rate being in a target heart rate zone), that the user is sufficiently ready to perform a first strenuous workout segment (e.g., class segment #3). The workout system 145 initiates the class segment #3.

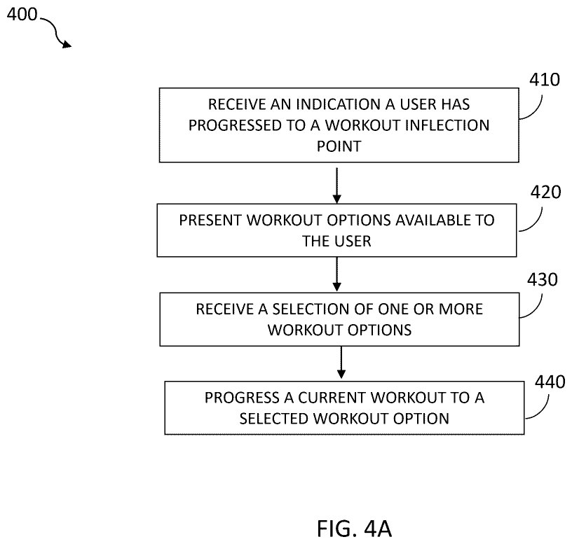

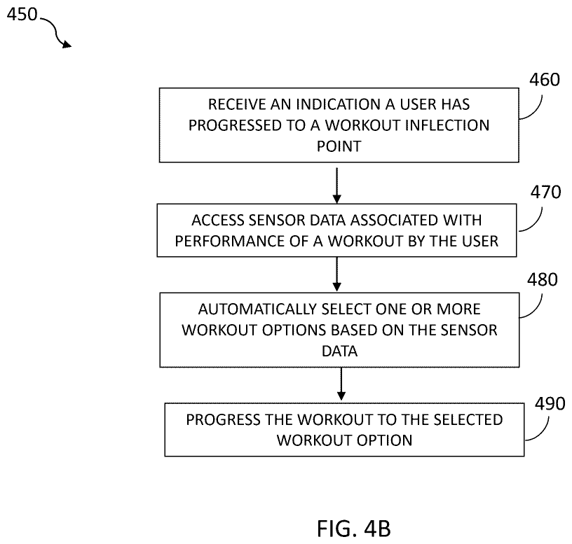

Thus, as described herein, the workout system 145 may perform various processes and/or methods when enabling users to select workout paths during and workout and/or automatically selecting and/or modifying workout paths for users during their exercise activities. FIG. 4A is a flow diagram illustrating an example method 400 for modifying a workout for a user of a connected fitness platform. The method 400 may be performed by the workout system 145 and accordingly, is described herein merely by way of reference thereto. It will be appreciated that the method 400 may be performed on any suitable hardware.

In operation 410, the workout system 145 receives an indication that a user, performing a workout, has progressed to an inflection point within the workout. For example, the modification module 210 may receive an indication of the trigger event 205 within a workout performed by a user, such as any event or occurrence during a workout, during a workout segment, and/or during a time period between workout segments.

In some cases, such as when the workout includes an exercise class presented to a user via a device associated with the user, the workout system 145 may determine a playback of a current class segment of the exercise class has reached a timestamp within a multimedia stream of the exercise class that is associated with the inflection point.

In operation 420, the workout system 145 presents workout options available to the user and associated with the workout. For example, the output module 230 may identify, select, and/or present available or candidate workout options (e.g., workout segments or class segments) via an interface associated with the user.

In some cases, the workout system 145 may identify, via the metadata within the multimedia stream of the exercise class, one or more candidate workout options for presentation to the user at the inflection. For example, the metadata may include pointers or tags associated with the content 155 within the content database 155 and/or content 160 within the voice database 165.

The workout system 145 may select various available or candidate options for the user. For example, the workout system 145 may select and/or display candidate workout options that include class segments of the exercise class that are part of a path of class segments for the exercise class, workout options based on a current performance of the user measured at the inflection point within the workout, and so on.

In operation 430, the workout system 145 receives a selection of one or more of the presented workout options. For example, the output module 230 may receive an indication that the user has selected an option presented (e.g., visually or in audio form) by the workout system 145 during the workout.

In operation 440, the workout system 145 progresses the workout to the selected one or more workout options. For example, the output module 230, following the various workout paths depicted in FIG. 3, may initiate a workout segment selected by the user and/or based on the user's performance of one or more previous workout segments.

FIG. 4B is a flow diagram illustrating another example method 450 for modifying a workout for a user of a connected fitness platform. The method 450 may be performed by the workout system 145 and accordingly, is described herein merely by way of reference thereto. It will be appreciated that the method 450 may be performed on any suitable hardware.

In operation 460, the workout system 145 receives an indication that a user, performing a workout, has progressed to an inflection point within the workout. For example, the modification module 210 may receive an indication of the trigger event 205 within a workout performed by a user, such as any event or occurrence during a workout, during a workout segment, and/or during a time period between workout segments.

In some cases, the trigger event 205 may be based on the user's performance metrics or other data (e.g., heart rate, effort level, and so on). For example, the workout system 145 may receive an indication when the user has performed below a certain level or threshold for one or more workout segments and/or below an expected/predicted level or threshold for the user.

In operation 470, the workout system 145 accesses sensor data associated with performance of the workout by the user at the inflection point. For example, the sensor data module 220 may access sensor data representing one or more workout metrics associated with the workout performed by the user (e.g., in response to the trigger event). The sensor data may include heart rate data and/or other biometrics for the user while performing a current workout segment of the workout (e.g., captured by a wearable device or other mobile device), performance metrics associated with the user performing a current workout segment of the workout (e.g., captured by the wearable device and/or an associated exercise machine), and so on.

In operation 480, the workout system 145 may automatically selecting one or more workout options within the workout for the user based on the sensor data, and, in operation 490, progress the workout to the automatically selected one or more workout options. For example, the output module 230 may select a path or branch of workout segments based on the user's heart rate being within a certain heart rate zone, a user's effort level being below a baseline effort level, the user's performance metrics being above a certain activity level (e.g., within a top X percentage of users), and so on.

### Examples of Modifying Voice Instructions During a Workout

The connected fitness platform, as described herein, may enhance a virtual coach and/or virtual coaching service to provide real-time instructions or prompts (e.g., natural language encouragement, feedback, instructions, and so on) to users performing exercise activities. While the instructions may be in audio form (e.g., presented as voice instructions by a virtual coach or instructor of a class), in some cases, the instructions may be adapted (or accessibility or other factors) and be presented in textual form or representations (e.g., closed-captioning, subtitles, and so on), such as via the user's mobile device, an AR headset or smart glasses, and so on.

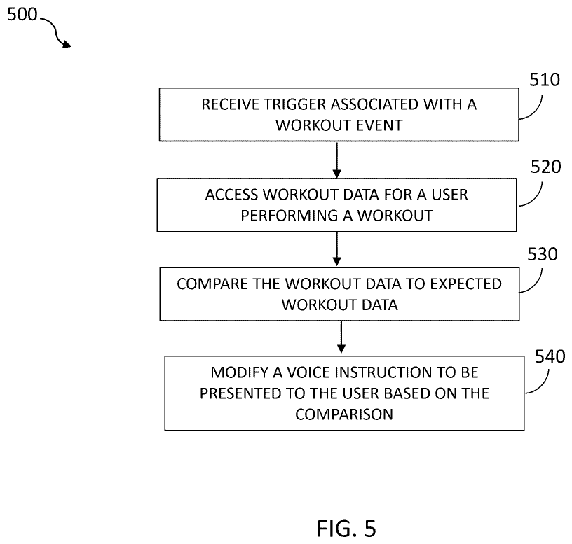

FIG. 5 is a flow diagram illustrating an example method 500 for presenting voice instructions to a user of a connected fitness platform. The method 500 may be performed by the workout system 145 and accordingly, is described herein merely by way of reference thereto. It will be appreciated that the method 500 may be performed on any suitable hardware.

In operation 510, the workout system 145 receives a trigger associated with a workout event. For example, the modification module 210 may receive an indication of the trigger event 205 within a workout performed by a user, such as any event or occurrence during a workout, during a workout segment, and/or during a time period between workout segments.

In operation 520, the workout system 145 accesses workout data for the user performing the workout. For example, the sensor data module 220 may access sensor data representing one or more workout metrics associated with the workout performed by the user, such as heart rate data and/or performance metrics associated with the user performing a current workout segment of the workout.

In operation 530, the workout system 145 compares the accessed sensor data to threshold sensor data associated with the voice instruction. For example, the output module 230 may determine the accessed sensor data does not satisfy threshold sensor data (e.g., a user's heart rate is not within an expected heart rate zone or heart rate zone associated with a current or next workout segment).

In operation 540, the workout system 145 modifies a voice instruction to be presented to the user based on the comparison. For example, the output module 230 may generate, using an LLM associated with an instructor or coach providing the voice instruction, a modified voice transcript for the modified voice instruction. In some cases, the LLM associated with the instructor is an existing speech synthesis model for the instructor that is trained using exercise classes taught by the instructor within the connected fitness platform. Further, as described herein, the workout system 145 may present the modified voice instruction after completion of a workout segment and/or during a time between workout segments of a workout.

As an example, the workout system 145 can acquire, in real-time, metrics associated a pace for a user performing a series of track intervals within a workout. The workout system 145, using the pace information, can modify various voice instructions for a real instructor or coach (e.g., represented as a virtual instructor or coach), as follows:

The user runs the first interval too quickly (e.g., three seconds too fast), and the workout system 145 generates and presents a voice instruction to the user during a rest interval that states: “you went out too fast, try and dial it back by a few seconds, pal.” The generated instruction has been modified to reflect a current performance by the user (e.g., to slow down) while still being uttered with the usual speech patterns and/or idiosyncrasies of the coach (e.g., the use of “dial it back” and “pal”);

The user overcompensates and runs the next interval too slowly. The workout system 145 generates and presents a subsequent voice instruction to the user during a next rest interval that states: “ok, ok, you know that was too slow, let's meet in the middle for the next one,” which, again, may mimic how the coach/instructor actually speaks to their clients.

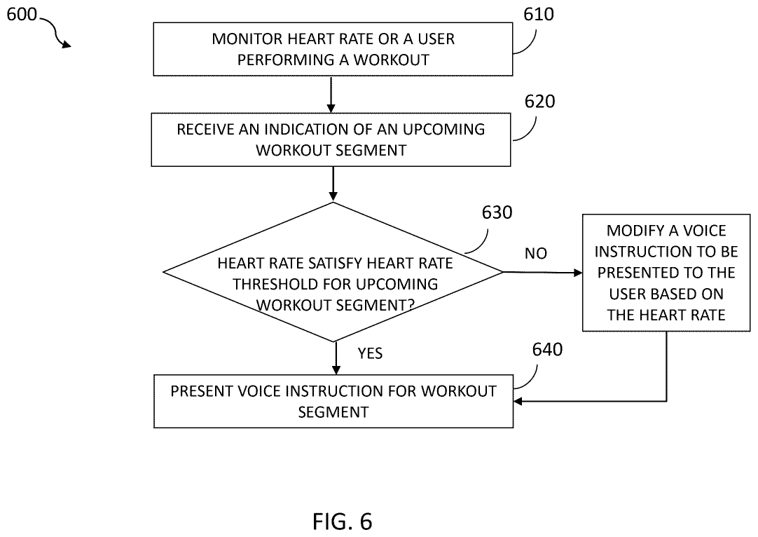

In some embodiments, the workout system 145 may modify a workout, as described herein, and generate a voice instruction based on the modification of the workout, such as to inform a user of the modification. FIG. 6 is a flow diagram illustrating an example method 600 for coaching a user of a connected fitness platform. The method 600 may be performed by the workout system 145 and accordingly, is described herein merely by way of reference thereto. It will be appreciated that the method 600 may be performed on any suitable hardware.

In operation 610, the workout system 145 monitors the heart rate of a user performing an exercise activity. For example, the workout system 145 may capture the heart rate and/or performance metrics for the user during a workout, such as at different intervals within the workout.

In operation 620, the workout system 145 receives an indication of an upcoming exercise segment within the exercise activity. For example, the workout system 145 may receive a trigger event and/or other indications of a subsequence exercise segment.

In operation 630, the workout system 145 determines whether the monitored heart rate satisfies a heart rate threshold for the upcoming exercise segment. When the heart rate satisfies the threshold, the workout system moves to operation 640, and presents a previously generated voice instruction for the workout segment.

However, when the monitored heart rate does not satisfy the threshold, the workout system moves to operation 650 and modifies the voice instruction to be presented to the user based on the heart rate (e.g., based on a comparison to the threshold heart rate). As described herein, the workout system 145 may generate, using an LLM associated with an instructor providing the voice instruction, a modified voice transcript for the modified voice instruction.

As an example, a user is performing the interval workout described herein. The workout system 145, via an application on the user's smart watch, monitors the user's heart rate during rest intervals. When the user's heart rate reaches a target rate during the rest interval (e.g., zone 2), the workout system 145, regardless of how long the user has been resting, may cause the user to begin a next sprinting interval, by generating and presenting the following voice instruction: “ok, pal, you've cooled down enough, next interval in 5, 4, 3, 2, 1 . . . go!”

As another example, the workout system 145 is monitoring the user's heart during an entire workout and comparing the heart rate, over the workout, to an expected heart rate for the user (e.g., based on previous workouts by the user). Before a final sprinting interval, the workout system 145 determines the heart rate has been above the expected heart rate (e.g., heart rate peaks or an average heart rate have been above norms) and generates/presents a voice instruction to end the workout early: “you seem pretty beat, let's cool down now.”

### Examples Scenarios for the Workout System

As described herein, the workout system 145 may enhance and/or modify workouts for users in real-time in a dynamic manner. The following scenarios provide example implementations across different exercise or fitness modalities.

In a first scenario, a user running on a treadmill is participating with other users in a streamed exercise class led by an instructor. After a difficult “hills” section, the class presents a graphical element that prompts the user with two choices: “Ready for more hills?” or “Ready to descend?” The user selects the first option. The workout system 145, as described herein, replaces the first part of a subsequent descent section with an additional hills section, and moves the user to the new hills section, before moving the user into the descent section at a later time.

In a second scenario, a bicyclist is riding a century (e.g., 100 miles) and has a goal to finish in under six hours. The workout system 145, monitoring the bicyclist's heart rate and performance metrics (e.g., distance over time) determines the user has fallen behind and is predicted to not achieve the goal, but their heart rate information indicates the bicyclist can ride faster. Based on the determination, the workout system 145 generates a voice instruction, as follows: “Listen, you are a bit behind, but you have it in you-increase your speed one mile per hour for the next 10 minutes and let's reassess.”

In a third scenario, a user is lifting weights, and their metrics are being tracked by weight machines (e.g., cable-based weight machines or motor-driven strength machines) and/or connected dumbbells. During a second to last set of a current movement (e.g., overhead press), the workout system 145 determines the user is fatiguing one rep less than normal (e.g., reaching failure, or close to failure, at 9 reps instead of a target 10 reps for a given weight). The workout system 145 automatically modifies the last set of the current movement to 6 reps and generates a voice instruction to inform the user, as follows: “that last set seemed tough, let's de-load a bit and just give me 6 good reps on the final set.”

Of course, the workout system 145 may be implemented in other scenarios and/or for other modalities, as described herein.

### Examples Embodiments of the Technology

As described herein, the systems and methods may enable, support, and facilitate modifications of workouts or exercise activities, as set forth in the following example embodiments.

In some embodiments, a system associated with a connected fitness platform comprises a modification module that receives a trigger event within a workout performed by a user and tracked by the connected fitness platform, a sensor data module that accesses sensor data representing one or more workout metrics associated with the workout performed by the user in response to the trigger event, and an output module that generates a modified workout based on the one or more workout metrics for the workout performed by the user.

In some cases, the output module modifies a path of workout segments presented to the user within the workout based on the one or more workout metrics not satisfying threshold metrics for the path of workout segments.

In some cases, the output module inserts a workout segment into a path of workout segments presented to the user within the workout based on the one or more workout metrics not satisfying threshold metrics for the path of workout segments.

In some cases, the output module modifies a next workout segment of a path of workout segments presented to the user within the workout based on the one or more workout metrics.

In some cases, the trigger event is a selection by the user of one or more candidate workout segments of the workout, and wherein the one or more candidate workout segments are selected based on the one or more workout metrics.

In some cases, the trigger event is the user completing a current workout segment within the workout.

In some cases, the sensor data includes a heart rate for the user while performing a current workout segment of the workout.

In some cases, the sensor data includes a performance metric associated with the user performing a current workout segment of the workout.

In some cases, the user performs the workout via an exercise machine associated with the connected fitness platform, and wherein the one or more workout metrics are performance metrics captured by the exercise machine.

In some cases, wherein the one or more workout metrics are performance metrics captured by a wearable device associated with the user that is communicatively coupled with the connected fitness platform.

In some embodiments, a method comprises receiving an indication that a user, performing a workout, has progressed to an inflection point within the workout, presenting workout options available to the user and associated with the workout, receiving a selection of one or more of the presented workout options, and progressing the workout to the selected one or more workout options.

In some cases, the workout includes an exercise class presented to a user via a device associated with the user and receiving the indication that the user has progressed to the inflection point within the workout includes determining a playback of a current class segment of the exercise class has reached a timestamp within a multimedia stream of the exercise class that is associated with the inflection point.

In some cases, presenting workout options available to the user and associated with the workout includes identifying, via metadata within the multimedia stream, one or more candidate workout options for presentation to the user at the inflection point.

In some cases, the candidate workout options include class segments of the exercise class that are part of a path of class segments for the exercise class.

In some cases, the one or more candidate workout options are identified at least in part on a current performance of the user measured at the inflection point within the workout.

In some cases, the workout includes an exercise class presented to a user via a mobile device associated with the user, and presenting workout options available to the user and associated with the workout includes presenting one or more user selectable options to the user via the mobile device associated with the user.

In some cases, the user is performing the workout via an exercise machine, and wherein a display of the exercise machine presents the one or more user selectable options to the user.

In some embodiments, a non-transitory, computer-readable medium whose contents, when executed by a computing system, causes the computing system to perform a method, including receiving an indication that a user, performing a workout, has progressed to an inflection point within the workout, accessing sensor data associated with performance of the workout by the user at the inflection point, automatically selecting one or more workout options within the workout for the user based on the sensor data, and progressing the workout to the automatically selected one or more workout options.

In some cases, the user is performing the workout via an exercise machine, and the sensor data includes one or more performance metrics captured by the exercise machine and associated with the performance of the workout by the user.

In some cases, the sensor data includes a heart rate for the user during the performance of the workout.

### Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system associated with a connected fitness platform, the system comprising:
a modification module that receives a trigger event within a workout performed by a user and tracked by the connected fitness platform;
a sensor data module that accesses sensor data representing one or more workout metrics associated with the workout performed by the user in response to the trigger event; and
an output module that generates a modified workout based on the one or more workout metrics for the workout performed by the user.

2. The system of claim 1, wherein the output module modifies a path of workout segments presented to the user within the workout based on the one or more workout metrics not satisfying threshold metrics for the path of workout segments.

3. The system of claim 1, wherein the output module inserts a workout segment into a path of workout segments presented to the user within the workout based on the one or more workout metrics not satisfying threshold metrics for the path of workout segments.

4. The system of claim 1, wherein the output module modifies a next workout segment of a path of workout segments presented to the user within the workout based on the one or more workout metrics.

5. The system of claim 1, wherein the trigger event is a selection by the user of one or more candidate workout segments of the workout, and wherein the one or more candidate workout segments are selected based on the one or more workout metrics.

6. The system of claim 1, wherein the trigger event is the user completing a current workout segment within the workout.

7. The system of claim 1, wherein the sensor data includes a heart rate for the user while performing a current workout segment of the workout.

8. The system of claim 1, wherein the sensor data includes a performance metric associated with the user performing a current workout segment of the workout.

9. The system of claim 1, wherein the user performs the workout via an exercise machine associated with the connected fitness platform, and wherein the one or more workout metrics are performance metrics captured by the exercise machine.

10. The system of claim 1, wherein the one or more workout metrics are performance metrics captured by a wearable device associated with the user that is communicatively coupled with the connected fitness platform.

11. A method, comprising:
receiving an indication that a user, performing a workout, has progressed to an inflection point within the workout;
presenting workout options available to the user and associated with the workout;
receiving a selection of one or more of the presented workout options; and
progressing the workout to the selected one or more workout options.

12. The method of claim 11, wherein the workout includes an exercise class presented to a user via a device associated with the user, and
wherein receiving the indication that the user has progressed to the inflection point within the workout includes determining a playback of a current class segment of the exercise class has reached a timestamp within a multimedia stream of the exercise class that is associated with the inflection point.

13. The method of claim 12, wherein presenting workout options available to the user and associated with the workout includes identifying, via metadata within the multimedia stream, one or more candidate workout options for presentation to the user at the inflection point.

14. The method of claim 13, wherein the candidate workout options include class segments of the exercise class that are part of a path of class segments for the exercise class.

15. The method of claim 13, wherein the one or more candidate workout options are identified at least in part on a current performance of the user measured at the inflection point within the workout.

16. The method of claim 11, wherein the workout includes an exercise class presented to a user via a mobile device associated with the user, and
wherein presenting workout options available to the user and associated with the workout includes presenting one or more user selectable options to the user via the mobile device associated with the user.

17. The method of claim 16, wherein the user is performing the workout via an exercise machine, and wherein a display of the exercise machine presents the one or more user selectable options to the user.

18. A non-transitory, computer-readable medium whose contents, when executed by a computing system, causes the computing system to perform a method, the method comprising:
receiving an indication that a user, performing a workout, has progressed to an inflection point within the workout;
accessing sensor data associated with performance of the workout by the user at the inflection point;
automatically selecting one or more workout options within the workout for the user based on the sensor data; and
progressing the workout to the automatically selected one or more workout options.

19. The non-transitory, computer-readable medium of claim 18, wherein the user is performing the workout via an exercise machine, and wherein the sensor data includes one or more performance metrics captured by the exercise machine and associated with the performance of the workout by the user.

20. The non-transitory, computer-readable medium of claim 18, wherein the sensor data includes a heart rate for the user during the performance of the workout.
